(19) United States
(12) Patent Application Publication
(10) Pub. No.: US 2012/0283663 A1
(43) Pub. Date: Nov. 8, 2012

(54) Sheath for Protection of Medical Devices and Method of Use Thereof

(76) Inventor: REBECCA DELEGGE, MT. PLEASANT, SC (US)

(21) Appl. No.: 13/463,281

(22) Filed: May 3, 2012

Related U.S. Application Data
(60) Provisional application No. 61/481,769, filed on May 3, 2011.

(51) Int. Cl. A61M 25/00

(52) U.S. Cl. 604/264

(57) ABSTRACT

A protective sheath for protecting elongated medical devices positioned in, or through, the sheath while performing a surgical procedure. The sheath is formed of a thin, flexible material that provides and maintains a clean and sterile and reliable path for medical instruments to travel within. The device provides a barrier between a medical instrument, the orifice/opening in which the instrument enters the human body, and the path these instruments travel to other body organs or locations, and reduces unwanted translocation.

This application claims benefit of pending U.S. provisional application Ser. No. 61/481,769 filed May 3, 2011.

### BACKGROUND OF THE INVENTION

1. Field of the Invention

The present invention relates to sheaths for protecting medical devices. More particularly, this invention relates to flexible, disposable sheaths and methods of using the sheaths to provide and maintain clean and sterile medical devices and to provide a reliable path within the sheaths for the medical devices to travel.

2. Description of Related Art

Many medical devices, such as endoscopes, laryngoscopes, ultrasonic probes or similar elongated surgical instruments, pass through orifices or body openings to get to their final working location. These devices enter the body via the mouth, the anus, a vein or artery or other orifice. For example, in a procedure called NOTES (Natural Orifice translumenal endoscopic surgery) an endoscope enters the body through the mouth, it traverses the esophagus and stomach and enters the peritoneal cavity. The endoscope and other elongated medical instruments are exposed to body fluids in the GI tract such as saliva, gastric juices and bile, some of which are not sterile. The devices become contaminated with pathogens, such as bacteria, fungi, virus, as well as blood, vitreous and other contaminants or cells which may be harmless in one body area like the GI tract, but when moved to another body area (peritoneal cavity), can be harmful and even cause death. Introducing an infection with an endoscope positioned in a lung, bladder, or kidney, for example, may seriously impact a generally healthy patient; however, the impact is more severe on an immuno-compromised patient such as one suffering from AIDS. Further, even in a simple endoscopy of a patient with malignant cells may cause the translocation of these pharyngeal malignant cells to other portions of the GI tract or in the case of NOTES to other body systems. In addition, the pathogens and other contaminants can be transmitted to another patient during a subsequent procedure using the same instrument. Currently, sterility, when using procedures such as trans-oral peritoneal surgery, is achieved by cleaning the mouth, esophagus and stomach as well as possible prior to passing medical instruments and visualization tools, such as an endoscope, into the sterile thoracic, peritoneal or pelvic cavity. It is imperative that these cavities be maintained as bacteria, fungal, viral and contaminant free as possible.

One of the most important facets of these procedures is maintaining instrument sterility while moving the medical instrument from a body entry location to another body site. Another important facet is that the devices must be sterilized after each use. One way to avoid having to sterilize the devices is to provide a sheath which protects the devices from abrasion and minimizes contact of the device with the body cavity and the body fluids. One type of sheath is designed to fit snugly around the elongated medical device much like a condom. These types of sheaths are rolled up and placed over the distal end of the medical device and unrolled onto the shaft of the medical device. These sheaths typically fit tightly to the shaft of the medical device while others are secured in place with an elastic band or the like. In either case the medical device is not free to move within the sheath.

There is need for a thin, non-constraining sheath through which medical instruments, such as an endoscopic feeding tubes and the like, can be placed as they pass from the mouth into place in the periodontal cavity or through such other location in the body as a particular procedure may dictate. This sheathing should prevent translocation of pharyngeal or esophageal cancer cells to the abdominal wall. In addition, bacteria, fungi, viruses and other contaminants from the mouth, esophagus and stomach would not be translocated to the abdominal wall during placement of the medical instrument. Such sheaths should be dimensioned so that the elongated medical device may be freely moved therein and should have a means for grasping the proximal end of the sheath to maintain the sheath in place or to move the elongated shaft of the medical device within the sheath.

### SUMMARY OF THE INVENTION

It is therefore a general object of the present invention to provide a disposable, flexible sheath compatible with elongated medical devices that enters a body cavity which eliminates concerns for translocation of pathogens and the need for sterilization of the device between uses, while at the same time permits communication between the body cavity and the device.

Another object of the present invention is to provide a sheath for use with an elongated medical device whose interior is dimensioned such that the medical device may be freely moved with the sheath.

Yet another object of the present invention is to provide a sheath having a grasping means at the proximal end for placing or moving the sheath along the shaft of the medical device.

Still another object of the present invention is to provide a method of using the sheath to provide and maintain a clean and sterile medical device and to provide a reliable path within the sheath for the medical devices to travel.

The objectives are achieved by providing a sheath for covering and protecting portions of an elongated medical device. The present invention is a protective sheath that protects a medical device by placing the medical device in or through a protective sheath. The sheath is a thin-walled flexible material that provides and maintains a clean/sterile and reliable path for medical instruments to travel within. The sheath provides a barrier between a medical instrument, the opening in which the instrument enters the human body, and the path the instrument travels to other body organs or locations, and reduces the likelihood of unwanted translocation.

Other objects features and advantages of the present invention will become apparent from the following detailed description of the invention taken in conjunction with the accompanying drawings.

### BRIEF DESCRIPTION OF THE DRAWINGS

Having described the invention in general terms, reference will now be made to the accompanying drawings, which are not necessarily drawn to scale, and wherein:

FIG. 1 is a perspective view of an embodiment of the disposable sheath of the present invention having a medical device in place within the sheath;

FIG. 2 is a perspective view of another embodiment of the present invention showing the distal end of the sheath having a tapered configuration and providing for an opening in the tip;

FIG. 3 is a view showing an embodiment of an anchoring ring or grasp in the form of two flaps located at the proximal end of the sheath;

FIG. 4 is a view of the anchoring ring or grasp of FIG. 3 showing the grasp flaps in an open position; and

FIG. 5 is a view showing another embodiment of an anchoring ring or grasp in the form of a ring located at the proximal end of the sheath.

### DESCRIPTION OF THE PREFERRED EMBODIMENT(S)

The present inventions now will be described more fully hereinafter with reference to the accompanying drawings, in which preferred embodiments of the invention are shown. This invention may, however, be embodied in many different forms and should not be construed as limited to the embodiments set forth herein; rather these embodiments are provided so that this disclosure will be through and complete and will fully convey the scope of the invention to those skilled in the art. Like numbers refer to the elements throughout.

The sheath comprises a flexible elongated body portion having a distal end and a proximal end and defining an opening extending from the proximal end to the distal end. The proximal end of the sheath is capable of receiving the distal end of a medical device through the opening. In one embodiment the distal end has a sealed tip, while in another embodiment the distal end permits an orifice to be formed in situ though which the distal tip of the medical device may be passed, such as by using the medical device to puncture the distal end after entry into a site.

At the proximal end of the sheath there is an anchoring ring or grasp for holding the sheath in position or for moving the sheath along the shaft of the medical device. In one embodiment the anchoring ring or grasp comprises a pair of flaps of sheath material as an extension of the proximal end of the sheath that extends outwardly from the body opening and is of sufficient size to be grasped by the doctor. In another embodiment the anchoring ring or grasp is a ring or gasket attached to the proximal end of the sheath through which the shaft of the medical instrument passes. The invention also provides methods for using the sheath.

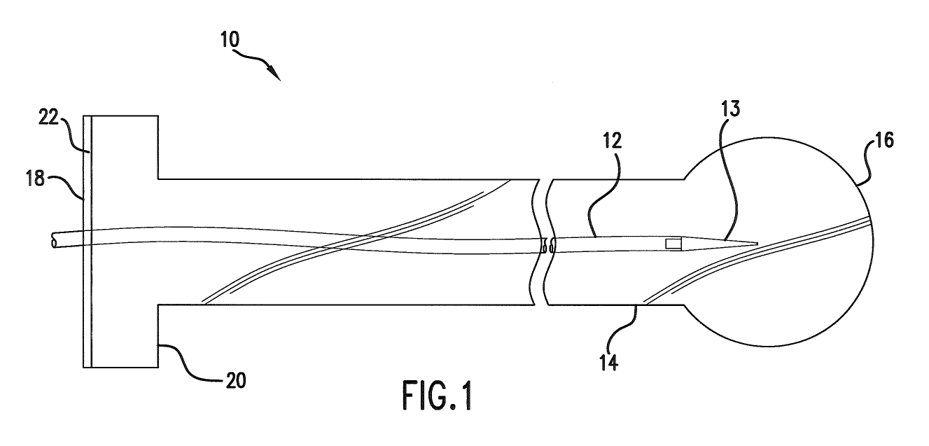

As shown in FIG. 1 there is provided sheath 10 according to an embodiment of the invention showing a portion of medical device 11, such as a feeding tube, endoscope or other medical instrument having an elongated shaft 12 in place within a sheath 10. The distal end of the medical instrument has a tip 13. The sheath 10 comprises a flexible elongated body portion 14 having a distal end 16 and a proximal end 18. The proximal end 18 defines an opening 22 extending from the proximal end 18 to the distal end 16 to accommodate the shaft 12, or a portion thereof, of the medical device 11. An anchoring ring or grasp 20 is preferred to be located at the proximal end 18 connected to an opening 22.

The elongated body portion 14 is preferred to be dimensioned so that its interior cross-sectional area is slightly larger than the elongated shaft 12 of the medical device 11 that it is intended to cover. This will insure that the elongated body portion 14 surrounds the shaft 12 of the medical device 11. The medical device 11 may be freely moved within the elongated body portion 14, while preventing contamination of the device 11 by pathogens or translocation of cells. The outside of the sheath 10, if desired, may be lubricated, such as with a surgical lubricant which may be a lubricating jelly.

Sheath 10 is made of a thin-walled, flexible material to form a barrier between the sterile area inside of the sheath and the contaminated areas of the body cavity outside of the sheath. The sheath may be made of polyurethane, polyethylene, polytetrafluoroethylene (PTFE), latex or a similar flexible material having the properties required for the sheath. The sheath material may be hydrophilic. Preferably, the thin-wall material is smooth on both sides. The preferred wall thickness does not exceed 0.007 inches (7 mil). In one embodiment, the elongated body portion of the sheath comprises two thin transparent sheets bonded at their side edges with a heat seal that is impermeable to pathogens. In another embodiment the elongated body portion comprises a tube that is impermeable to pathogens.

A flat piece of material folded and heat sealed on one side may be used to form the sheath. The seam may be turned inward so that it does not irritate an anatomical lumen through which it passes.

The elongated body portion 14 of the sheath 10 is preferred to be long enough to cover that portion of the medical device 11 within the patient. In an embodiment of the invention for use through the mouth, the sheath is not less than the length from the patient's mouth to the gastroenterological junction. This distance is typically about 12 inches. It is understood that the length of the elongated body portion 14 will vary depending upon the procedure and medical instrument used.

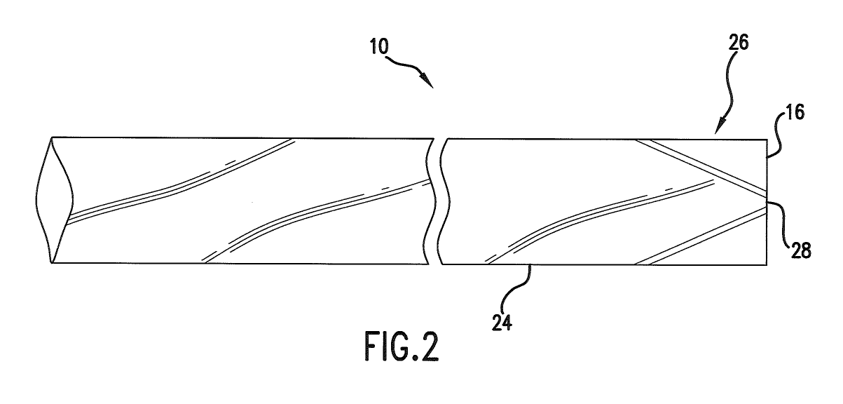

In the embodiment of the sheath 10 shown in FIG. 1, the distal end 16 of the elongated body portion 14 is an enlarged bulb, or bulbous, shape, having generally the shape of a balloon. This embodiment shows the distal end 16 of the elongated body portion 14 as having a tip that is sealed. It should be understood that the shape of the distal end may take a variety of shapes and sizes. For example, in the embodiment shown in FIG. 2, the distal end 16 of the elongated body portion 14 has a taper in the opening or orifice of the elongated body. The taper may be formed by heat sealed seams 27, 29. An orifice may be 28 formed through the tip at the distal end. The taper assists in guiding the instrument through the orifice in this embodiment. A feeding tube or other medical instrument may be pushed through the orifice 28 in the tip to accommodate the medical instrument for performing portions of a medical procedure. Generally, is preferred to be sized according the orifice size so that additional material from a sheath that is too large does not interfere with medical instrument manipulation.

The tip at the distal end has a weakened portion. By weakened portion, it is meant a portion of the distal end of the body of the sheath that may be easily pierced or punctured by the end of the elongated medical device as the elongated medical device slides relative to the elongated body. This weakened portion may be formed of a material that is not as thick as the remainder of the elongated body, or is sealed in a weakened fashion so that the elongated medical device or instrument will pierce or puncture the distal end of the elongated body, or is otherwise compromised so that the distal end is easily penetrated by sliding the distal end of the medical device thought the elongated body as the elongated body is in place, such as by holding by the anchoring ring. The length of the medical device or instrument exceeds the length of the elongated body so that the medical device or instrument is pushed through the elongated body after the construct has been transported to the site of the medical procedure.

In order to maintain the sheath 10 in its desired position, or to move the sheath along the shaft of the medical device, an anchoring ring or grasp 20 is provided. The grasp 20 is located at the proximal end 18 of the elongated body portion extending out of a body cavity and integrally formed with the opening 22 in the elongated body portion 14. The attending physician or assistant may simply hold the grasp to keep the sheath in place and guide the medical instrument accordingly or move the sheath along the shaft of the medical instrument.

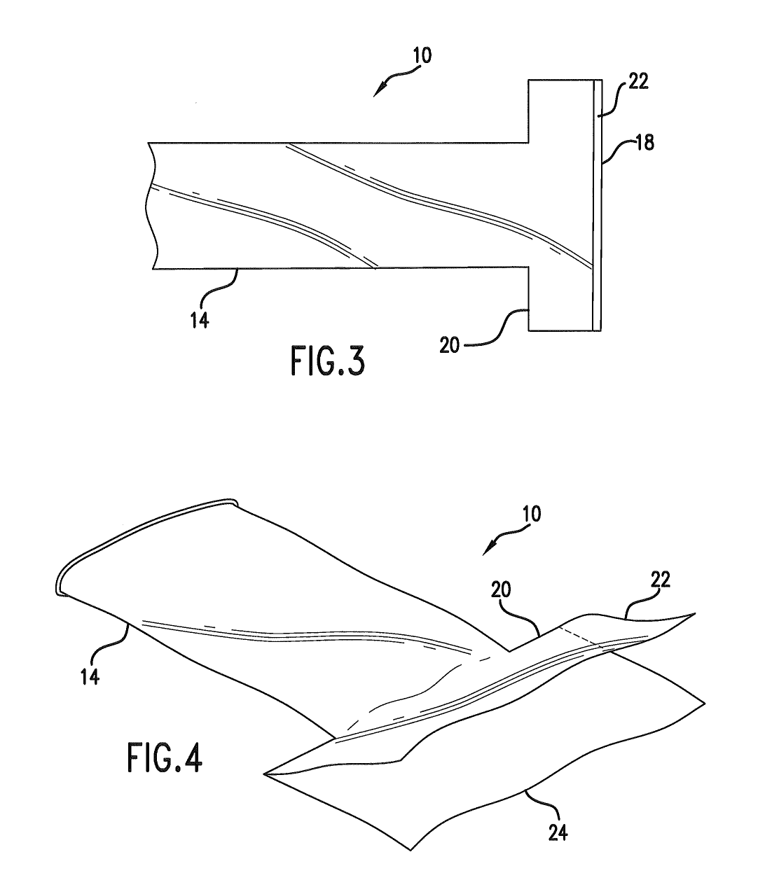

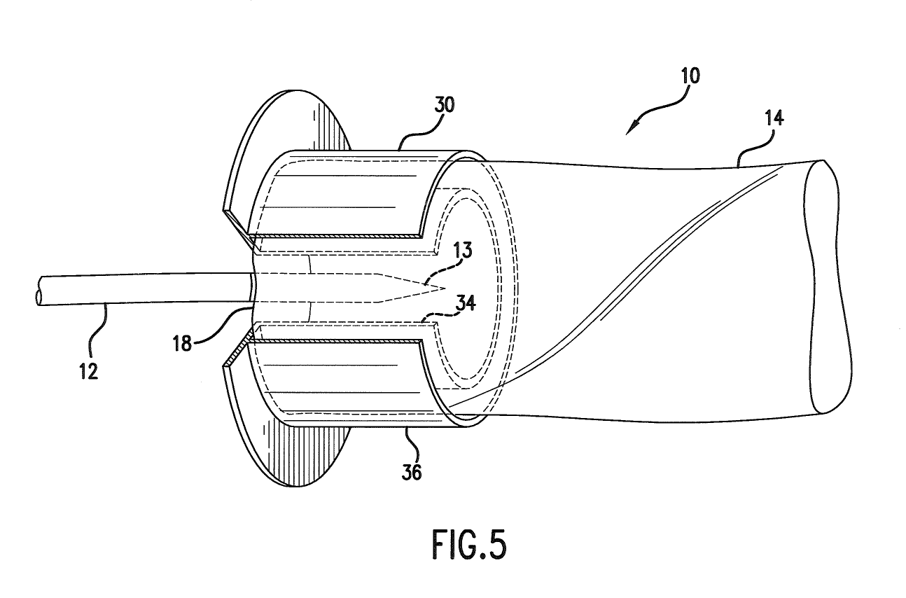

In one embodiment, that shown in FIG. 3 and FIG. 4, the anchoring ring or grasp 20 comprises a first flap or extension 24 and a second flap 26 or extension located on an opposite side of the elongated body. The flaps extend outwardly from the elongated body portion, and may be grasped by the user and moved to change the position of the medical device. The flaps 24, 26 may be extensions of the thin-walled, flexible sheath material and are preferably wider than the elongated body portion 14 for ease of grasping. In this embodiment, the overall structure of the sheath has a “T” shape.

In the embodiment shown in FIG. 5, the anchoring ring is an annular ring or gasket 30 located at or near the proximal end 18 of the elongated body portion 14 of the sheath 10. This annular ring is useful for holding the sheath in position, particularly when the elongated medical instrument slides or moves within and relative to the elongated body of the sheath. A feeding tube, endoscope or other medical device 11 is passed through the ring 30 into the elongated body portion 14. The anchoring ring of this embodiment has a ring portion 30 with circular inner wall 34 and a circular outer wall 36 attached thereto. The elongated body portion 14 is affixed to the ring 30 by attachment between the walls 34, 36. The circular inner wall 34 and the circular outer wall 36 may be connected to each other by rim 32.

In use according to an embodiment, the sheath is anchored in the mouth and the elongated medical instrument passes within the sheath. The enlarged anchoring ring or grasp may be sized to be too large to pass through the mouth, and assists in holding the elongated body of the sheath in position, and limits movement of the elongated body as the medical instrument passes within it. Similar sizing of the anchoring ring or grasp may be provided for other applications so that the anchoring ring or grasp is outside of an orifice and lumen, whether anatomical or surgically formed, for limiting travel of the elongated body. When the moving instrument reaches the end of the stationary elongated sheath, it punctures the sheath. The sheath that has passed through the mouth is “dirty,” while the scope tip that has just punctured through the sheath is maintained as clean by the sheath. A clean instrument is in contact with the tissue which is to be cut or otherwise violated. In addition, the sheath may be a puncturable package, where one end of the sheath is perforated or weakened relative to the remained of the elongated body of the sheath to allow the elongated instrument to more easily puncture and pass through the sheath after unsterile or risky anatomy is passed. This device and process allow the introduction of a clean instrument through a dirty orifice. In the case of malignant disease, a clean instrument may be passed into an area where a cut is to be made. This avoids malignant seeding or bacterial translocation at the site.

In yet another embodiment, parallel sheathing procedures permit access to be gained without compromising sterility. The sheath may be formed of a co-extrusion comprising two or more lumens, with one lumen for an endoscope and others for other instruments. A sheath having a multiple lumens facilitates the achievement of certain tasks. For example, endoscopes commonly have one or two lumens to pass instruments. The ability to have a multi lumen sheath to pass multiple instruments and visualization tools is a major asset.

The invention also provides a method for using the sheaths. The sheath is a conduit for moving a sterile medical instrument that is particularly useful in trans-oral peritoneal surgery. The sheath provides a path for medical instrumentation to travel within a sterile field. The sheath is inserted as required through the mouth or other body orifices, and subsequently may enter the chest, peritoneal or pelvic cavities as required.

When using the procedures from outside of the body, access is made to the esophagus, stomach or intestine within the sterile sheath 10 such as that shown in FIG. 2. The sheath abuts the gastrointestinal tissue, and the esophageal, gastric or intestinal wall is punctured with a trocar or other cutting device. A port or other fixation/portal device is placed into the puncture to serve as an anchoring device and to connect the sheath to the port or tissue wall, and to provide entry to the thoracic, peritoneal or abdominal cavity. This device eliminates clumsy gastrointestinal decontamination procedures, and provides a reliable, sterile entry to the thoracic, peritoneal or pelvic cavities.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A sheath for covering portions of a medical device having an elongated shaft comprising:
an elongated body formed of a thin, flexible material that is impermeable to pathogens, the elongated body comprising a distal end and a proximal end, wherein the proximal end has an opening that is formed to receive a distal end of a medical device having an elongated shaft within the elongated body, wherein the opening extends through the elongated body to a closed distal end, the elongated body comprising an interior cross sectional dimension that is larger than a portion of the medical device that the sheath is constructed to cover, wherein the elongated body is constructed and arranged to cover the portion of the medical device while permitting the portion of the elongated medical device to slidably engage the elongated body;
a tip formed on the distal end of the elongated body, the tip comprising a weakened portion; and
an anchoring ring comprising an opening that communicates with the opening of the elongated body, the anchoring ring extending from the distal end of the elongated body, wherein the anchoring ring is enlarged relative to the elongated body;
wherein the elongated medical device has a length that exceeds the length of the elongated body, and wherein, in use a distal end of the elongated medical device is substantially in contact with an interior of the distal end of the elongated body, and the elongated medical device is transported to a site within the elongated body, and upon reaching the site, the distal end of the elongated medical device is pushed through the weakened portion of the tip of the elongated body and the distal end of the elongated medical device exits the distal end of the elongated body while the elongated body is held in position by the anchoring ring.

2. The sheath according to claim 1, wherein said anchoring ring comprises two flaps that extend outwardly from the elongated body portion that are constructed and arranged to be grasped by a user to change a position of the elongated medical device.

3. The sheath according to claim 1, wherein said anchoring ring comprises a ring at the proximal end of the elongated body, the anchoring ring comprising an inner wall and an outer wall, said inner wall and outer wall being connected to each other at one end by a rim, said elongated body portion being attached to said rim between said inner wall and said outer wall.

4. The sheath according to claim 1 wherein said anchoring ring is formed integrally with said body portion as a unitary member.

5. The sheath according to claim 1 wherein the tip formed on the distal end of the sheath is bulbous and is enlarged from the elongated body.

6. The sheath according to claim 1 wherein the tip formed on the distal end of the sheath is tapered to interior cross sectional dimensions that are smaller than the interior cross section dimension of the elongated body.

7. The sheath according to claim 1 wherein the tip formed on the distal end of the sheath has an orifice therein constructed and arranged for exit of the distal end of the elongated medical devices.

8. The sheath according to claim 1 wherein said elongated body portion has a wall thickness that does not exceed 0.007 inches.

9. The sheath according to claim 1 wherein said elongated body portion comprises two transparent sheets bonded at their side edges with a heat seal that is impermeable to pathogens.

10. The sheath according to claim 1 wherein said sheath is formed of a material from a group consisting of polyurethane, polyethylene, PTFE, PEEK, FEP, PET, EVA, PFA, rubber, latex and silicone.

11. The sheath according to claim 1 wherein the overall shape of the sheath is a T shape.

12. The sheath according to claim 1 wherein an area inside the elongated body of the sheath is sterile and is maintained as sterile by the sheath during use.

13. The sheath according to claim 1 wherein an interior of the distal end of the elongated body tapers to form the tip comprising the weakened portion.

14. A method for protecting an elongated medical device during a surgical procedure comprising:
placing a sheath formed of a thin, flexible material that is impermeable to pathogens over a distal end of a shaft of an elongated medical device, said having a distal end and a proximal end, the proximal end comprising a longitudinal opening, wherein the opening extends through an elongated body of the sheath the proximal end to the distal end, wherein the opening is constructed and arranged to receive the distal end of the shaft of the elongated medical device, and wherein the sheath has a anchoring ring formed near the proximal end of the elongated body, wherein the anchoring ring communicates with the proximal end of the elongated body; and
holding said anchoring ring and inserting the elongated medical device into the sheath and sliding the elongated medical device into the sheath to a desired position relative to the sheath;
holding said anchoring ring and positioning the sheath containing the elongated medical device for use in a medical procedure.

15. The method for protecting an elongated medical device during a surgical procedure according to claim 14 wherein said anchoring ring comprises two flaps that extend outwardly from opposite sides of the elongated body near the proximal end of the elongated body.

16. The method for protecting an elongated medical device during a surgical procedure according to claim 14 wherein said anchoring ring comprises a ring positioned near the proximal end of the elongated body, the anchoring ring comprising an inner wall and an outer wall, said inner wall and outer wall being connected to each other at one end thereof by a rim, said elongated body portion being attached to said rim between said inner wall and said outer wall.

17. The method for protecting an elongated medical device during a surgical procedure according to claim 14, wherein the elongated medical device slidably punctures the sheath after the elongated medical device is transported to a site of the medical procedure.

18. The sheath according to claim 14, wherein an area inside the elongated body of the sheath is clean or sterile and is maintained as clean or sterile by the sheath during use.
